(19) United States
(12) Patent Application Publication
(10) Pub. No.: US 2003/0144877 A1
(43) Pub. Date: Jul. 31, 2003

(54) Hierarchical network system for disseminating medical, drug and diagnostic information and guidance

(76) Inventors: David R. Goldmann, Wynnewood, PA (US); Thomson M. Kuhn, Alexandria, VA (US)

Correspondence Address: OBERMAYER REBMANN MAXWELL & HIPPEL LLP, 1617 JOHN F KENNEDY BLVD, 19TH FLOOR, PHILIDELPHIA, PA, 19103, US

(21) Appl. No.: 10/060,062

(22) Filed: Jan. 29, 2002

(51) Int. Cl. G06F 17/60; A61B 5/00

(52) U.S. Cl. 705/2; 600/300

(57) ABSTRACT

A hierarchical system for providing medical information: a control unit for setting forth navigation and for providing a plurality of medical guidance information for use by a medical professional; means for assisting the medical professional in navigating and searching disease specific hierarchical information in a database; means for distributing the hierarchical information to said medical practitioner; and means for rating the hierarchical information.

### FIELD OF THE INVENTION

The present invention is directed to online computer systems. In particular, the present invention is specifically directed to online computer systems that can be used to handle the dissemination of medical information, medical advice, disease diagnosis, guidance and recommendations.

### BACKGROUND OF THE INVENTION

The Internet or World Wide Web is one of the most critical technological developments of the last decade. The Internet has provided vast economic opportunities for numerous businesses and industries to vastly expand the number and quality of their services. One of the earliest and fastest emerging areas of Internet activity has been in providing rapid, up-to-the-minute business information. To date, a number of patents have issued on Internet related systems that cover a wide array of business information and electronic commerce (e-commerce) applications.

One of the largest uses of the Internet has been in the field of providing medical related information. While the prior art discloses various mechanisms for disseminating medical information, there is no prior art system which comprehensively follows the diagnostic processes of a physician, and which provides, at a single location, an intuitive navigation driven system for providing disease diagnosis, drug, non-drug and treatment recommendations and advice.

There is a long felt need for a system that would more completely provide information to medical practitioners and the like. Such a system would enable Internet users and medical practitioners to obtain comprehensively delivered medical information and diagnostic assistance.

A number of patents have issued directed to systems for disseminating medical information. U.S. Pat. No. 6,322,502, for example, is directed to a medical information system that receives patient data and information from various sources and displays such information in a variety of formats for use by members of a medical team in a hospital, clinic, or office. The system 10 includes a primary display 12, and an associated display controller 14 and System storage device 16. The controller 14 is coupled to a primary interface unit 20. Keyboard and/or pointing device 22, scanner 24, audio input and/or output device 26 and printer 28 are all coupled by way of interface 20 to the display controller 14. Access to selected subsets of patient information is provided by user selection of specific data sets identified by job function selection icons. Multiple types of patient data are selectively displayed simultaneously, and to multiple remote users.

U.S. Pat. No. 5,918,208 is directed to a managed care expert system which provides a graphical, interactive computer system which accepts user input relating to contract variables for a plurality of alternative contract scenarios, consults a database of national and locality-specific utilization data, performs a utilization and revenue analysis for both commercial and Medicare-age beneficiaries, and provides a synthetic fee schedule for comparing the likely revenue under capitation for a plurality of services to revenue for those services under a current reimbursement scenario. The system of the invention, in its preferred embodiment, enables a physician or other health care professional to use a broad array of assumptions to forecast utilization of medical procedures and estimated revenue per procedure under multiple capitation scenarios.

U.S. Pat. No. 5,915,240 is a Medical Lookup Reference computer system for accessing medical information over a network. The system partitions the functioning of the system between a client and server program in an optimal manner to assure synchronization of a master medical information database on the server with a local medical information database on the client. This minimizes the use of network resources, and allows new types of medical information to be easily included in the system. A server site on the network maintains a description of the medical information, as well as the most current and up-to-date medical reference information. The client program maintains a local database that is automatically synchronized over the network with revisions and new medical information, and provides a user with an interface to fully review the information in the database. The system also uses a context-sensitive call facility so that users of the Medical Lookup Reference program can easily get further expert assistance about the medical topic. The call feature uses the network connection to establish a conversation between the user and a person at a help site specified by the type of medical information they are currently referencing. Once a connection is established, the system allows the user to engage in a conversation with the person at the help site, and a record of the conversation can be saved in a database for auditing purposes.

Finally, U.S. Pat. No. 5,779,634 is directed to a medical information processing system for supporting diagnostic interpretation, featuring a data storage unit for storing an interpretation image and interpretation reference images to which a doctor will refer to interpret the interpretation image. A data-loading unit loads the interpretation reference images from the data storage unit into a workstation unit according to a predetermined priority order. The data-loading unit loads the images into a workstation that is selected from the workstation unit according to workstation vs. interpretation examination modality information. A diagnostic information creation unit creates diagnostic information relative to the image by inputting the doctor's findings or computerizing with a computer unit. Positions of abnormalities and degrees of the abnormalities are determined, and positions in association with the images are calculated.

A diagnostic information comparing unit compares the diagnostic data and shows differences between the diagnostic information as time-sequential abnormality change data. The diagnostic information output unit outputs the diagnostic information or results of comparing the diagnostic data and superimposes the contents of the time-sequential abnormality change data on the associated image.

A number of popular and well-known Internet websites have also provided medical related information including DRKOOP.COM and WEBMD.COM. Such websites and web applications merely provide generic information on medical on medical issues. In short, they merely provide basic information and do not provide comprehensive medical advice, diagnostic information and treatment tools.

It would be desirable to provide a comprehensive physician information and education resource which improves over prior art systems for providing medical related information.

It is therefore a principal object of the present invention to provide a comprehensive system for providing medical information and data.

It is another object of the present invention to provide a system by which medical information can be provided to medical professionals and practitioners and which is set out in an intuitive, hierarchical manner which can be easily navigated.

It is a further object of the present invention to provide a site and system whereby end users can obtain important medical information about various diseases, their diagnosis and treatment.

It is yet a further object of the present invention to provide a system by which a medical expert can place a request for medical information and the medical information can be provided with a high level of confidence in its currentcy, correctness and completeness and in which the data are graded based upon reliability.

It is still a further object of the present invention to provide a comprehensive medical information system.

It is yet a further object of the present invention to provide a comprehensive medical information system that can be linked from electronic medical records (EMR).

These and other objects of the present invention will become apparent from the detailed description and from the following summary, detailed description and claims.

### SUMMARY OF THE INVENTION

The present invention is directed to medical information systems. In a most preferred embodiment, the present invention is directed to a hierarchical system for disseminating comprehensive medical information. The present invention is designed to work at a plurality of levels within the healthcare workflow. The present invention provides the health professional with relevant alerts and “things to consider” for a specific patient's disease management when electronic medical records (EMR) are available.

In an individual or group practice, the invention provides quick answers when needed as well as patient centered information that can be printed on site and emailed or faxed to the client. The invention can further be utilized for Continuing Medical Education (CME) credits, enabling physicians to improve the pedagogical experience for certification and re-certification, while not taking additional time out of their schedule.

Unlike traditional medical information sources, the present invention is centered on specific, bottom-line action statements with links to explanations, sources, rationale, etc. With traditional medical information sources, specific, bottom-line action statements, if they exist at all, are buried in pages of discussion and commentary. The basic structure of the system is identical from topic to topic. Information structure is organized to match the clinical workflow. A critical advantage of the present invention is its speed: the physician gets to the relevant bottom-line action statements faster and with significantly less cognitive overhead.

In one embodiment, the invention comprises a hierarchical system for providing medical information comprising a control unit for setting forth navigation and for providing a plurality of medical guidance information for use by a medical professional; means for assisting the medical profession in navigating and searching disease specific hierarchical information in a database; means for distributing the hierarchical information to said medical practitioner; and means for rating the hierarchical information.

In a further embodiment, the invention is a hierarchical system for providing medical information to medical practitioner comprising: a control unit for setting forth system navigation and for providing a plurality of rated medical guidance information stored in a database; means for assisting a medical professional in obtaining disease specific hierarchical information from the database; means for distributing hierarchical information to said medical practitioners.

In still a further embodiment, the invention is a method for generating medical information in a global computer system comprising the following steps: authoring a plurality of medical related guidance statements related to specific diseases and rating said information based upon at least one reliability criterion; placing the plurality of statements in an accessible database as a module and linking thereto a plurality of additional information and data related to the guidance statements; and providing end users with access to statements and modules via a user interface.

Finally, the invention is a method for generating medical information for use in a global computer system comprising the following steps: authoring and pre-storing a plurality of medical related guidance statements related to specific diseases and providing for each a qualitative rating based upon the evidence supporting the statement; placing the guidance statements within an accessible database as modules and linking thereto a plurality of additional information and data related to the guidance statements; and providing end users with access to statements and modules via a user interface such that the guidance statements, additional information and data are instantly accessible.

### BRIEF DESCRIPTION OF THE FIGURES

FIG. 1 is a block diagram of the operational system of the present invention.

FIG. 2 is a more detailed block diagram of the present invention.

FIG. 3 is a further diagram of the database of the present invention.

FIG. 4 illustrates the origination of the data flow from origination to right-of-access.

FIG. 5 illustrates the operational pyramid between content and navigation.

FIG. 6 illustrates the organization of a disease based module as used in the present invention.

FIGS. 7-19 illustrate a series of input screens for use by medical personnel in conjunction with a more preferred embodiment of the present invention.

### DETAILED DESCRIPTION OF THE PREFERRED EMBODIMENT

The present invention is directed to a system in which medical care providers can access, directly, hierarchical information related to diseases, diagnosis and drug therapy via a database. A critical feature of the present invention is the provision of an intuitive, navigation based system for providing comprehensive disease and treatment information at a single location. The present invention is thus specifically directed to a system whereby medical information and a variety of comprehensive diagnostic and disease information can be provided by a global computer network and in which specific guidance can be provided to end users.

The present invention is designed to work at a plurality of levels within the healthcare workflow. The present invention provides the health professional with relevant alerts and “things to consider” for a specific patient's disease management when electronic medical records (EMR) are available. In an individual or group practice, the invention provides quick answers when needed as well as patient centered information that can be printed on site and emailed or faxed to the client. The invention can further be utilized for Continuing Medical Education (CME) credits enabling physicians to improve the pedagogical experience for certification and re-certification, while not taking additional time out of the physician's schedule

While the present invention is being described in the context of a system using a personal computer, the manner of the particular end user device is not critical to the present invention. The present invention may be used with any system that connects to the Internet or uses other IP transport methods. The end user device can comprise any end user device that can connect to a network such as a wireless device, palm pilot, PDA, end user workstation or hand-held device.

The technical and operational background of one embodiment of the present invention is now described. Over the past fifteen (15) years, personal computers have become relatively powerful and inexpensive and have gained widespread use in a significant number of homes and businesses. With a modem, personal computers can communicate with other computers through communication networks and access many resources on the so-called “Information Super Highway.” Companies such as America Online, CompuServe and Prodigy, which traditionally provided so-called “content” over proprietary networks, have begun to provide access by personal computer users to an expansive international network of computer networks known as the Internet.

As is well known by those skilled in the art, the World Wide Web is a graphical sub-network of the Internet. With common “Web Browser” software such as Mosaic, Netscape Navigator, or Microsoft Explorer, end users may easily access Internet information and services on the World Wide Web. A web browser handles the functions of locating and targeting information on the Internet and displaying the information provided by the Web Server. The World Wide Web utilizes technology called “Hyper-Text” to organize, search and present information on the Internet. Using a web browser, the end user can select a word (“Hyper-Text word”) from a view document and be linked to another document featuring information related to the word. The present invention is thus designed, in one embodiment, to be utilized on the World Wide Web or Internet, although the present invention is equally applicable to other network environments. As noted above, the present invention is similarly related to user interfaces which are not computers such as palm pilots, wireless and cellular devices.

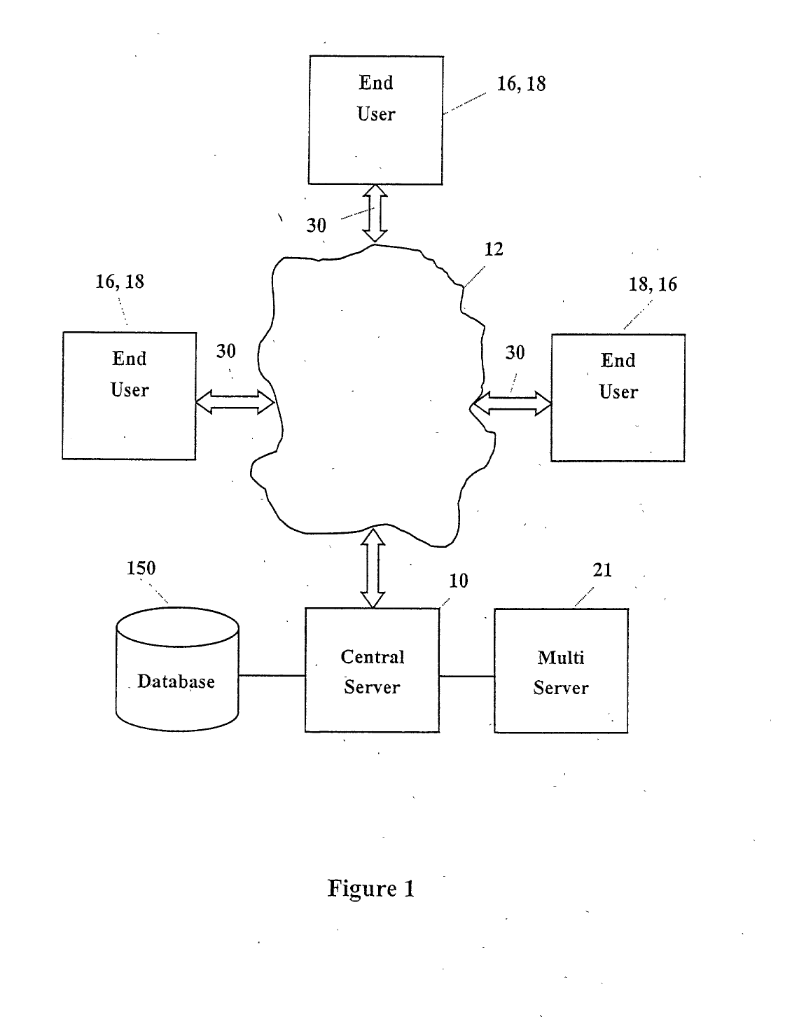

Referring to FIG. 1, a preferred embodiment of a system for the present invention is now disclosed and shown. As will be discussed herein, the present invention is directed to a comprehensive hierarchical system for providing medical information and guidance. A preferred embodiment comprises a central computer server 10 connected by a computer network 12 to remote end user stations 14. The central server connects to a database 150, which, as will be discussed herein, contains a plurality of medical guidance based information, statements and data which may be generated or authored by experts.

In a preferred embodiment, end user stations comprise a plurality of end users 16, 18. End users 16, 18, are defined herein as individuals linked to the system who may comprise medical practitioners, medical care providers and medical specialists. For purposes of this disclosure, a medical practitioner is defined as an individual who desires professional information and the like and wishes to utilize the system of the present invention.

Users 16, 18 are linked with the central computer server 10 via a transport medium 30. End users 16, 18, in a most preferred embodiment, will be linked via a global computer network 12 such as the Internet or Worldwide web, but other embodiments including LANs, WANs and Intranets, fulfill the spirit and scope of the present invention.

The end user devices 16, 18 will typically comprise any device that connects to the system via the Internet or other IP transport methods and includes, but is not limited to, such devices as televisions, computers, hand-held devices, cellular phones, land based telephones, wireless electronic devices and any device which uses a transport medium 30. Non-limiting examples of a transport medium 30 applicable for use in the present invention comprise any backbone or link such as an ATM link, FDDI link, satellite link, cable, cellular, twisted pair, fiber optic, broadcast wireless network, the internet, the world wide web, local area network (LAN), wide area network (WAN), or any other kind of intranet environment such as a standard Ethernet link. In such alternative cases, the client will communicate with the system using protocols appropriate to the network to which that client is attached. All such embodiments and equivalents thereof are intended to be within the scope of the present invention.

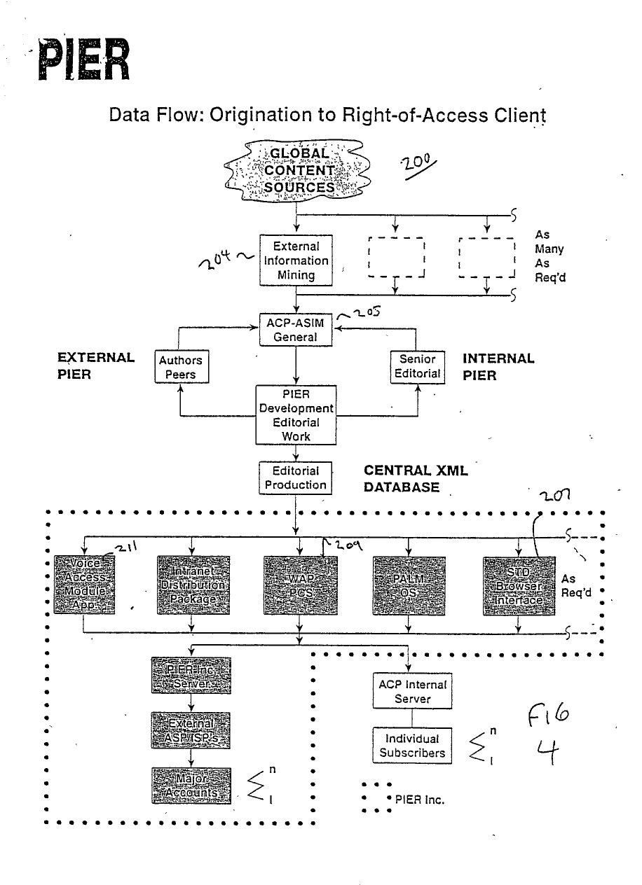

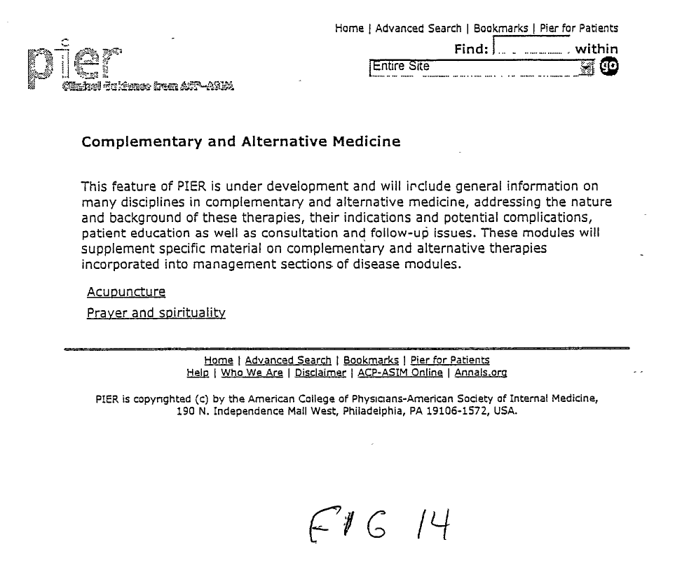

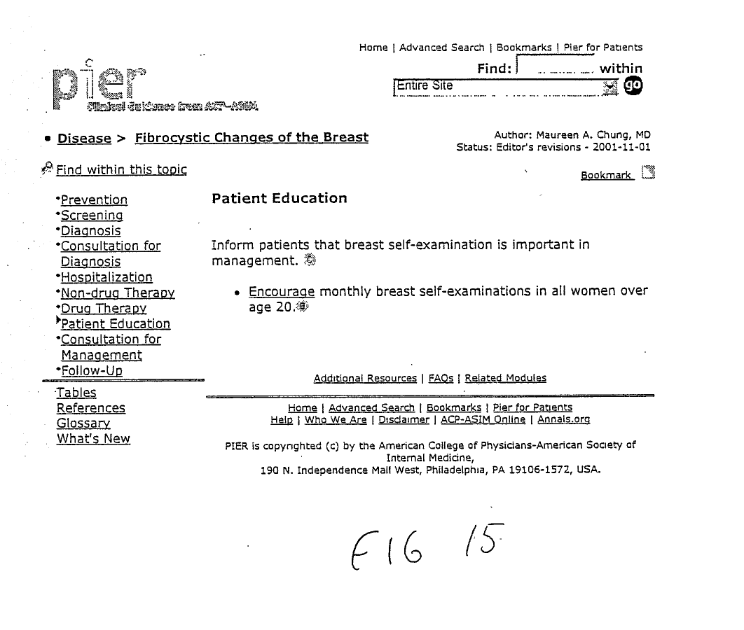

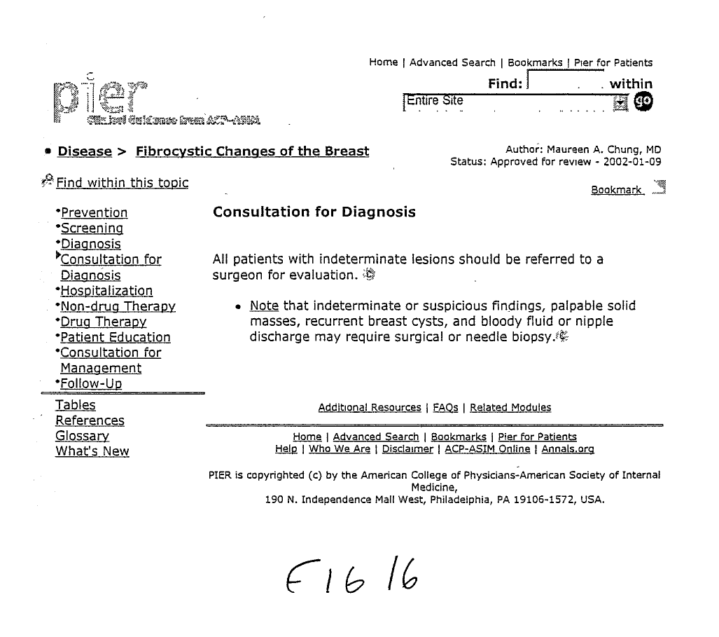

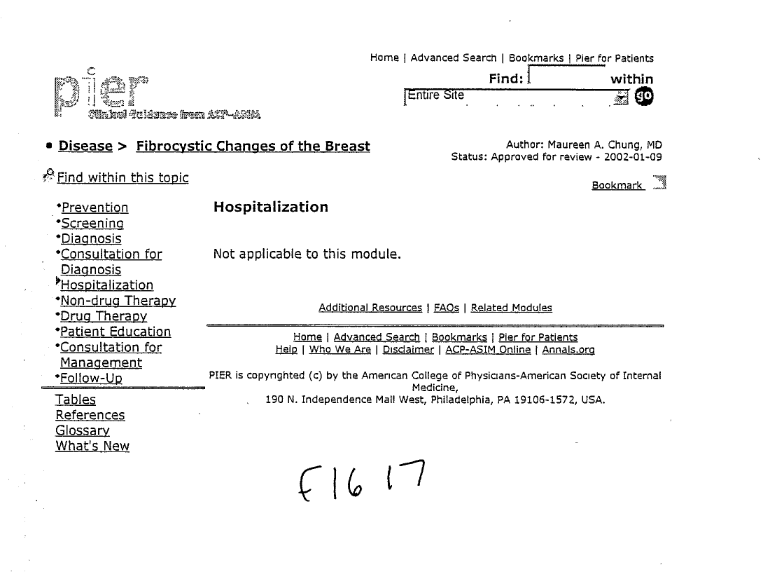

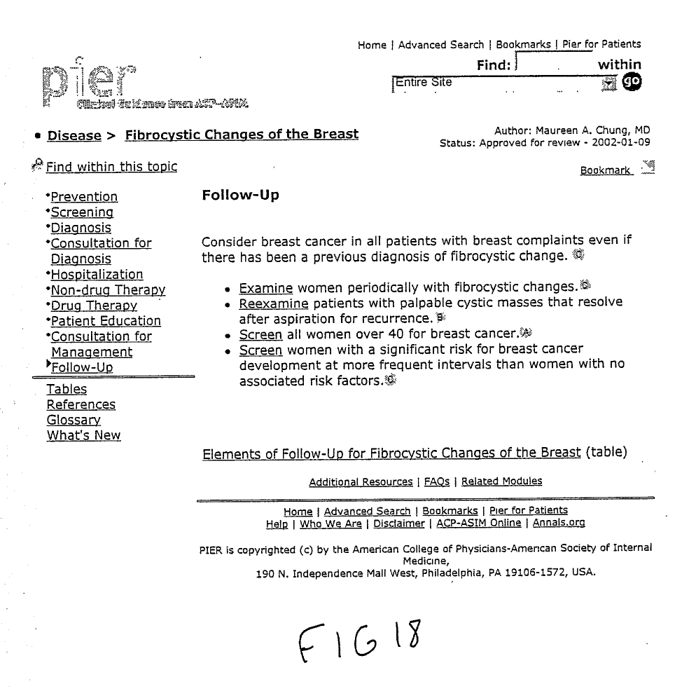

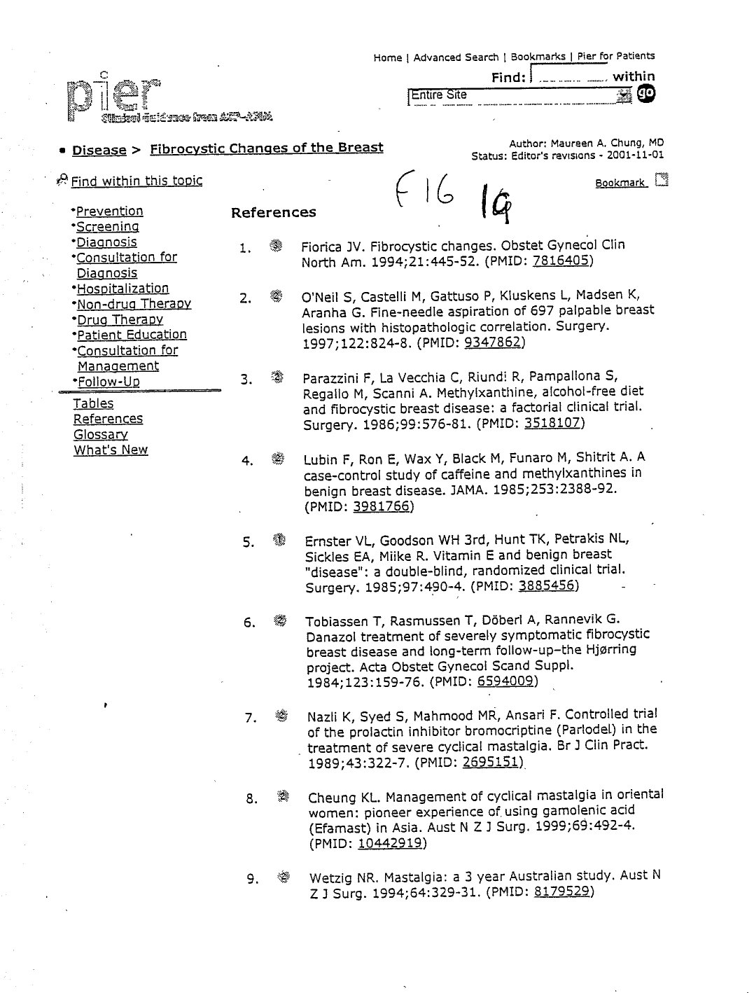

Referring again to FIG. 1, the present invention may comprise a multi-server 21 environment which comprises a computer system in accordance with the present invention that allows the multiple end users 16, 18 to communicate with the system and system clients. Through communication link and transport medium 30, end users 16, 18 will receive data entries which must be correctly identified and confirmed and who are linked to the central server 12, preferably by a customizable interface to be described in greater detail below with respect to FIGS. 4 to 19.

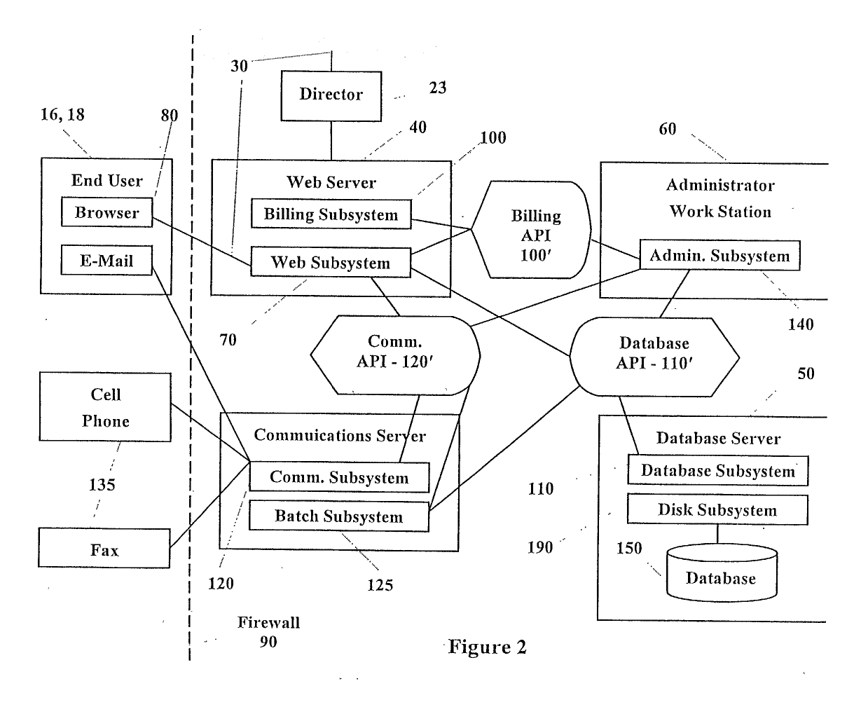

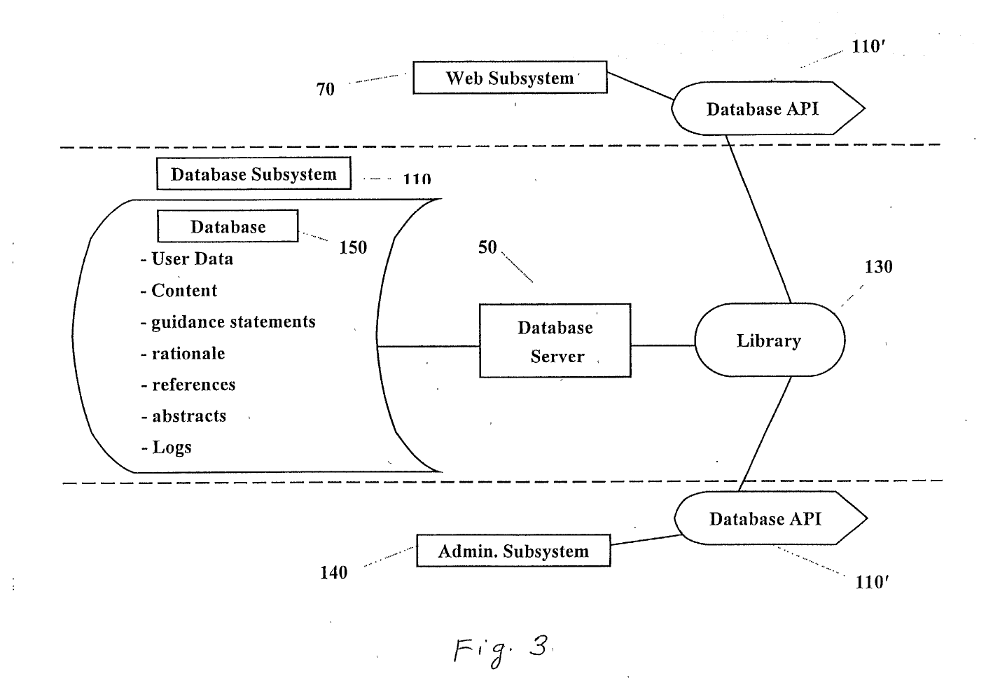

Referring to FIGS. 2 and 3, the central server and database systems of the present invention are now shown and described in greater detail. A local director 23 routes signals through the system to the various servers, to be described below, and to and through transport medium 30 to end users 16, 18. The system preferably includes two primary servers, a web server 40 and a database server 50 which may operate using such database platforms as SQL server or Oracle. Hence, in one embodiment the SQL server may run SQL server database management software from Microsoft Corporation. Alternatively, the server can further comprise an Oracle database server. The system further includes an administrative workstation 60 or system that provides the administrative capabilities and monitoring for the system under the control of an administrative subsystem 140.

The administrative workstation 60 allows administrators or other operators to perform routine operations that affect the entire system. Such operations include, but are not limited to, administering the accounts of end users 16, 18 monitoring the traffic through the system, tabulating user uses, printing reports, updating end users, performing backups and maintaining the programs that comprise the overall system. In addition, the administrative station 60 is utilized to update and add records, guidance statements, sub-statements and medical information into the system to be described in greater detail below.

A web subsystem 70 is responsible for all interactions with a web browser 80 in the end user devices 16, 18 and serves as the end user interface to the system. All interactions between the end user devices 16, 18 and the database subsystem occur through the web subsystem 70. Internet Information Server 200 (IIS) by Microsoft Corporation is an exemplary web server software system 70 in accordance with the present invention, although the present invention is in no way limited to this system. The expression of the user interface presented to end users 16, 18 in their client devices may be implemented as HTML or other high level computer language or technology, and may be displayed in a standard web browser. Typically, the interface will be presented as a website presentation such as www.acponline.org, owned by the American College of Physicians-American Society of Internal Medicine, Inc. assignee of the present invention.

All systems listed above are preferably communicated via an Ethernet 100 base T network and a switching hub. In addition, a second isolated network segment will preferably exist between the web server 40 and the external communications hardware (e.g. internet router). Such a system will keep external traffic isolated from the internal network, as well as provide a dedicated connection between the web server 40 and the Internet for maximum throughput. The systems will have an initial configuration of random access memory for the web server 40 and preferably at least 128 megabits for the database server 50, both having the capability to expand.

The web server 40 is the point of entry to the entire system. The system determines the identity of the user 16, 18 and makes appropriate decisions while serving web pages to the end user 16, 18. The web server 40 sends HTML or other high level computer language to the end user work stations 16, 18, validates passwords, sends logging and transaction information to the database server 50, and performs logical operations, thus behaving as a transactional server.

As noted above, in one embodiment, the server operating system may be a Windows NT server, a multi-platform operating system provided by Microsoft Corporation. The Sun Microsystems Solaris is an alternative embodiment. The server typically includes IIS, which is a completely integrated Internet application platform. IIS includes a high-performance web server, an application development environment, integrated full-text searching, multi-media streaming and site management tools. The security infrastructure is integrated within the server, thus enabling an easy-to-maintain and highly-secure web development and deployment environment.

The operators of the system may create, delete and update account information by utilizing the administrative subsystem 140 in administration workstation 60. A billing subsystem 100 may be incorporated. As will be discussed below, the operator will use the administrative workstation to add and edit specific medical information and guidance statements, sub-statements, rationale and evidence.

Database 110, communication 120 and billing 100 subsystems execute essential services for the other parts of the system, and will therefore have well-defined application program interfaces (API) 110′, 120′, 100′, as is well recognized by those with skill in the art. The system will preferably be protected for the Internet by a “firewall” 90 which is a safety precaution, and important with respect to the present invention due to the sensitive and confidential nature of the information in the database. As will be discussed below, firewall 90 plays an important and critical role in the present invention because of the confidentiality of the data associated with some applications of the present invention.

In a preferred embodiment, the database subsystem 110 stores all pertinent information pertaining to stored medial guidance information, rationale, references, user accounts, administrator accounts, as well as general dynamic system information. All interactions with the database subsystem 110 are performed through a database API 110′ which may define the interface to a library of stored procedures 130. These are used to implement high-level database functions and to shield the details of the database implementation from the other subsystems. The database subsystem 110 is preferably implemented using a database server 50 and database system.

A preferred system for use in the present invention utilizes XML to store content and documents. Extensible Markup Language (XML) is a markup language that provides a format for describing structured data. This facilitates more precise declarations of content and more meaningful search results across multiple platforms. In addition, XML is enabling a new generation of Web-based data viewing and manipulation applications. By storing data and content in XML, the system delivers content into multiple devices and interfaces from a single master files that conform to a custom-designed schema. It is to be appreciated that while one embodiment of the present invention is being described in the context of XML, other systems and technologies, known to those skilled in the art, are equally applicable.

It is to be stressed that the XML documents can be stored in and retrieved from any sort of database or document or file storage system. The critical feature is the hierarchical design and the unique schema of the invention. The web server 40 will delivers standard HTML pages to the users by transforming the content from its XML source format into a combination of HTML, JavaScript, and other technologies or formats. This transformation may be performed in batch, so that the completed HTML files are accessed by the server 40 when requested. Alternatively, the transformation could be performed in real time by the server when the content is requested.

The administration subsystem 140 provides an interface for operators and managers of the system to modify the database, print reports, view system data and log user comments and complaints. The administration subsystem 140 provides a collection of access forms, queries, reports and modules to implement the administration interface. Administrators typically will have the power within the system to force most actions. The administration subsystem 140 will interact with the communications, database and billing subsystems.

The communications subsystem 120 interfaced to a communications API 120′ will be used to email and contact end users 16, 18. End users 16, 18 may be notified by phone, fax, email or pager, or other communications devices that could be contacted by the system 135. End users 16, 18 may also have a password accessed section of a website where they can access information relevant to their activities and be provided with detailed reports.

Some portable telephones and pagers include email addresses and so may be contacted by the email system; other users have only phone numbers. Other interfaces may be utilized as the application so demands.

A batch subsystem 125 may periodically send out grouped notifications. It will access the database subsystem 110 to determine what notification is required, and uses the communication subsystem 120 to make those notifications. A group notification may comprise a special premium offered to end users 16, 18. The billing subsystem 100 will be used to verify and bill credit cards and communicate through the billing API 100′ to the administration subsystem 140, and potentially to an outside billing and verification service which could be used to perform the billing functions.

Referring to FIG. 3, the database server 50 which implements the database subsystem 110 of the present invention comprises a server that maintains all associated logging and transaction information for the system. Through the database 150 (which is backed up by a backup database for safety purposes), the database server 50 logs planner and provider setup and account creation information, maintains user account information, maintains account balances, produces and prints reports, hosts backup operations and performs statistical calculations for the entire system.

The database server 50 is preferably a dual processor computer microprocessor. Each connection to the database 150 and its associated work may be handled by a separate thread within the database server 50. It is anticipated that a dual processor machine is sufficient for the type and amount of transactions that it will be performing. However if it proves insufficient, the database can be “striped” to two or more machines to distribute the server load. As noted, in a most preferred embodiment, the content and documents are generated using XML. As noted, the database may store the guidance statements and sub-statements, rationale, evidence and other text.

The disk subsystem 190 of the database server may comprise a vulnerable and crucial server element. Due to the mission critical design of the subsystem, it is preferable to utilize a Level 5 RAID. Statistical calculations will be performed by the database server 50, along with other types of report generation. Specifically, IIS can log directly to an Open Database Connectivity ((ODBC) standard data source. This makes the availability of the data collected by the database server about client activity on the system more readily available and easier to process into logical reports.

In one embodiment, an operator workstation 60 may be used for administering the system. As the need for additional workstations arises, additional operator workstations can be added by adding additional computer systems, installing the administration software and connecting them to the LAN. Operator workstation machines preferably utilize a Windows operating environment manufactured by Microsoft Corporation.

With the above technical background, the present invention is now more fully described in accordance with the present embodiments as shown in FIGS. 4 to 7. In a most preferred embodiment, the present invention is specifically directed to a system which may be utilized online in accordance with the system of FIGS. 1 to 3 for providing physician decision support to physicians at the point-of-care, in order to improve patient safety, enhance outcomes, and increase the effectiveness of time-on-task, while also providing in context continuing education for the user. In a most preferred embodiment, the documents and content of the present invention have been created using XML (extendable mark-up language).

The system architecture is modeled to match the decision flow and work methodology of a physician or nurse. The system will typically be continuously updated and modular for installation in standalone, web, integrated EMR (electronic medical records), desktop, handheld, wireless, data and Interactive Voice Recognition applications. In one embodiment, the present invention provides an authoritative source of applicable “nuggets” of knowledge evidence based medicine available in ubiquitous form within the physician's normal workflow.

The present invention is thus structured to match the method a physician progresses in a diagnosis and uses the same terminology. The invention amplifies the physician's decision making, and is designed to obtain answers for the physician. It does not “search” for them.

In FIG. 4, the operation of the data flow is shown. The system will be driven by medical content obtained from global content sources 200 which may be mined 204 or, alternatively authored and edited by an internal staff 205. The information is then submitted for editorial review, stored in XML where it can be accessed by end users 16, 18 via a standard browser 207, wireless protocol 209, voice activated module 211 or intranet.

In operation, the system will be utilized in accordance with a webrowser 40 by the end users 16, 18. The layout of the interface may be accomplished using frames. In a web based presentation, the main content area contains a “depth” navigation system while a set of tabs immediately below allows “breadth” navigation special functions and supplementary content. Links scroll between content at a particular level and also open new windows containing deeper content, e.g., a rationale for guidance statements, figures and tables.

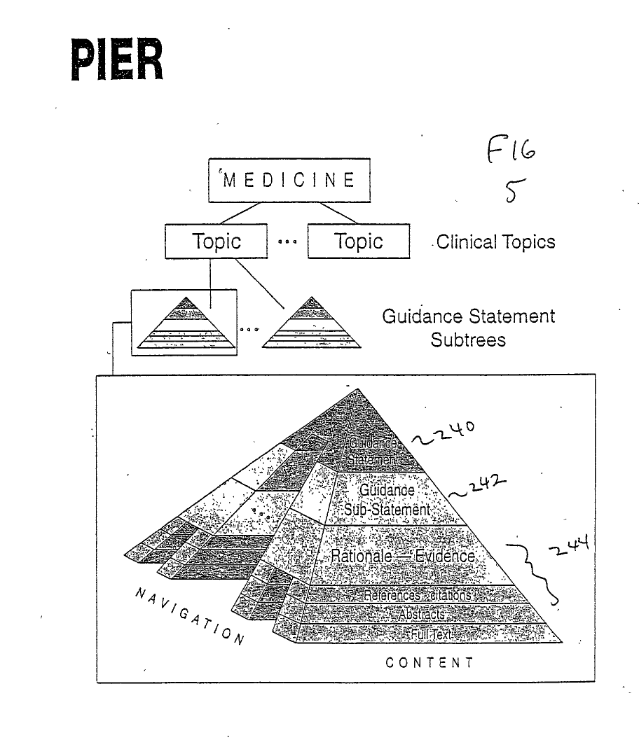

In FIG. 5 the layout of the content and disease modules of the invention is shown. Content will be supplied as a plurality of information modules that will typically be authored and edited by experts. In one embodiment, the modules themselves may be ordered in an alphabetical list, but the content within each will typically be arranged hierarchically.

In FIG. 5, guidance statements 240 represent the top level of the content tree. For each sub-statement 242 (a non-sub-stated guidance statement) a rationale explains the foundation for the guidance. Both the guidance and the rationale may be supported with references, linked directly to abstracts and/or full-text resources 244.

The guidance statements of the system will typically constitute the default tab for a selected module. This will contain the guidance statements, linked to sub-statements, and which allow content depth navigation. A tab contains a simulated search interface. The user selects to search either the “active” module or all modules for a word or phrase. The page is reloaded with a list of links to the matching content areas. In a preferred embodiment, the system contains a dated, chronological list of links to updated guidance statements containing all the new clinical information that has been added to the “active” module since its initial release. The system links to the full-text resources that triggered the need to update the content, so these are available directly as well.

This tab presents a menu of other full-text resources related to the topic of the “active” module. The content of these is compared with the content of the module and differences are presented in a tabular format. This area also presents an interface containing prepared live searches published and pre-populated with appropriate clinical terminology and user-selected criteria (overview vs. guideline, “recentness”, etc.) This tabulates all selected EMR (electronic medical record) orders and allows the user to deselect one, more, or all. Those features of this prototype that are not classifiable as “depth” or “breadth” navigation will be addressed in this section.

A critical feature of the present invention is that the guidance content is rated. That is, the degree to which guidance content is supported by evidence is a vital component of the usability of the content in a clinical setting. Each guidance statement and sub statement, within the present invention, is individually rated according to the evidence that supports it. The letters (A, B, C) are shown by each, either individually or in combination, e.g. AB indicates some of the statement is supported with level A evidence and some with level B. References themselves are rated on a three-point scale of their own (1,2,3) and are never used in combination since a particular paper or study, itself, defines the level of evidence. Classic mouse “rollovers” are used to dynamically present the level description for both the reference and statement evidence indicators.

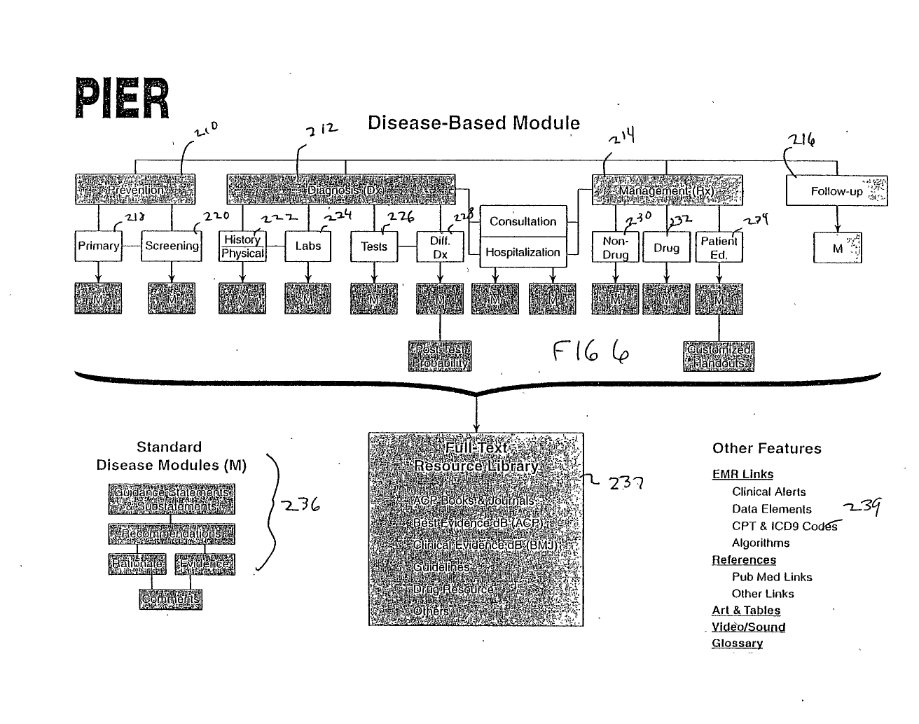

FIG. 6 illustrates the configuration of a disease based module as used in the present invention. The disease module is broken into four basic subsets; prevention 210, diagnosis 212, management 214 and follow up 216. Prevention is then broken into primary 218 and screening 220. Diagnosis is broken down into history 222, lab results 224, tests 226 and different diagnosis 228. Management is further broken down into non-drug 230, drug 232 and patient education 234. Each of these then leads to the disease modules 236. As shown in FIG. 6, the disease modules include guidance statements and sub-statements, recommendations and recommendation rationale, evidence and comments. The system will typically further incorporate a full text resource library 237 and other features and links 239.

Throughout guidance content navigation, including statements, sub-statements, rationale, and media, the user 16, 18 is presented with a set of bracketed links. By selectively clicking on these links, the user will register an order (pre-populated with appropriate dosage and other specific parameters) within the system. By clicking, for example, on a pre-selected button for “EMR Orders”, the user is presented with a list of all selected orders to date, with the option of clearing any or all of them.

In one embodiment, figures and tables constitute the media. In an alternative embodiment, video and audio files will complement the menu of media-enhanced content. Navigation to media elements is provided as an expanding list (labeled “Visuals” currently) in the leftmost frame below the list of modules.

Selection of an element opens a new window in which the figure or table is presented. In some cases, links from the media element will perform other functions such as adding an order to the EMR order tally or opening a citation window. In all cases, auxiliary windows of the same type (citation, media element, etc.) are re-purposed to avoid proliferation of windows. One window of each type, however, is accommodated.

A live search of the published medical data may be accessible via the other resources tab. End users can specify how recent they wish requested articles to be and whether they are interested in an Overview (defined as meta-analyses, systematic reviews or other overviews) or Guideline articles. These criteria, along with pre-populated appropriate clinical terminology, may be used to perform a query of a commercial database such as MEDLINE via an interface. Results are presented in a new window via an interface, so all functionality such as “show related articles” and abstract or, in specific cases, even full-text article browsing and printing is available.

Another feature directly available form a main content area is detailed drug information. User selection of a linked drug name in the guidance statements, sub-statements, rationale, and even certain media elements will produce a new window containing detailed information on the drug. Information includes description, uses, dosage and administration, preparations, and other clinically-relevant aspects of the drug.

The invention envisions numerous patient education materials via an expandable menu which may be labeled, for example “patient education” in the leftmost frame below the list of modules. These materials are being hosted external to the prototype and represent patient handouts that are deemed high quality and relevant by the module's editorial team. Production implementation of this feature has not yet been determined.

A “bookmarks” button at the lower left of the introductory screen opens a window containing a list and some options for managing the user's bookmarks. A previous version of the prototype allowed users to bookmark any guidance statement in order to return to it via these bookmarks. This functionality is only partially implemented in this prototype, though the capacity does exist as described in the technical specifications.

The present invention is now described in the context of the following example. The system is accessible via icon or linkage from some other information system. The user will be presented with a user screen via a web browser describing the system and providing a list of the most common (and most commonly mismanaged) clinical topics.

The user will then select a topic from the list and click on the topic name (“Diabetes mellitus, type 2”, for example). The user is then presented with a list of guidance statements for this topic as well as one or more Patient Education resources. By clicking on the arrow next to “Visuals”, all the figures and tables available for this module are disclosed. The user can then click on a guidance statement to see it expanded to show all its sub-statements (e.g. specific advice such as using stepped therapy to optimize glycemic control in patients with type 2 diabetes).

All sub-statements and the evidence levels of each are then noted. A sub-statement will provide additional advise such as (“Use pharmacological therapy if diet and exercise do not achieve the desired level of glycemic control”). The user can then click on each of the referenced tables to open them in a separate window. The user may then click on “metformin” link to open a window containing detailed drug information and then close the window; click on the “[order therapy]” link to prepare the order to be sent to an EMR; and click “ok” to acknowledge its registry. The user also may then click on the “Rationale” button to open a separate window containing rationale for the guidance provided in this sub-statement. The user may then click on the “references” tab at the bottom for a complete list of the references for this sub-statement and then, after noting the evidence indicators for each, the user may then click on an “Abstract” button to get an approved abstract of the reference. The window may then be closed by clicking the “Close Rationale” button. The system also includes a module search. This is activated by the user using the “Module Search” tab, in the search field, and clicking the “Search” button to run the search. The user clicks on the first link in the results to go to the match in the guidance statements content screen and then clicks on the “Module search” tab to return to the search area.

In a preferred embodiment, the presented content will largely comprise HTML files, utilizing style sheets for display-specific formatting via Cascading Style Sheets (CSS). Navigation and functionality are implemented through the use of JavaScript with code centralized in a JavaScript library (jslib.js). This source file is loaded into the main frameset upon application startup and all calls to the functions stored within are made within the context of the frame object model. Local, user-specific content storage, e.g., bookmarks and EMR orders, is achieved with cookies.

The content will be stored in a document management system that will manage access, check-in, check-out, workflow, and versioning. The document management system uses a Web interface. Authors, editors, and reviewers will be able to view, check-out, and check-in documents through their web browsers. Participants will be alerted to content needing their attention via automatically-generated emails.

The data may be tagged with so-called SNOMED RT terms. SNOMED RT is emerging as the most generally useful and widely implemented terminology in healthcare. SNOMED RT tags in the invention will be able to connect invention content to any healthcare system that implements SNOMED RT.

As noted above in FIGS. 4 and 5, a critical feature of the invention is the creation of content modules. In practice, expert authors will develop content modules using specialized templates accessible via the administrative subsystem. Such modules may be continuously modified and updated. The templates may require recent versions of Microsoft Word or other word processing program on a Windows platform. Templates are developed and modified by staff. Authors, associate editors and staff editors pass draft manuscripts back and forth through multiple cycles. It is expected that the documents will be transferred via email. The invention thus allows the automation of documents. Drafts can be stored in a secure document management system.

The workflow component is developed as an extension of the methodology used by physicians when undertaking an examination. When authoring-editing is complete, the modules will be handed-off to the production editors. Production editing will begin in Microsoft Word, using a system for storage. At some point, the contents of the module will be stripped from the underlying template and converted into XML. From that point on, further edition will be done in an XML-aware editing environment. As with authoring-editing, there will be BASIS license and custom development costs.

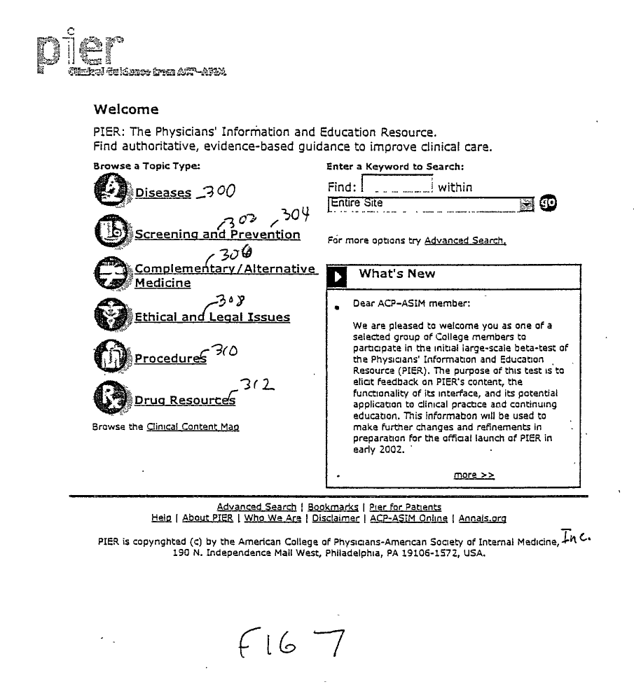

Referring now to FIGS. 7 through 19, the present invention is now more fully described in the context of a comprehensive website application which may be situated at a specific URL such as www.pieronline.org, the website of the American College of Physicians—American Society of Internal Medicine, Inc., the assignee of the present invention. The actual production website utilizes the principles set forth above and incorporates the navigation methodology described above.

As shown in FIG. 7, the initial page incorporates a series of icons covering diseases 300, screening 302 and prevention 304, complimentary/alternative medicine 306, ethical and legal issues 308, procedures and drug resources 312. Each of the pages is linked such as to provide a number of navigable methods for obtaining information.

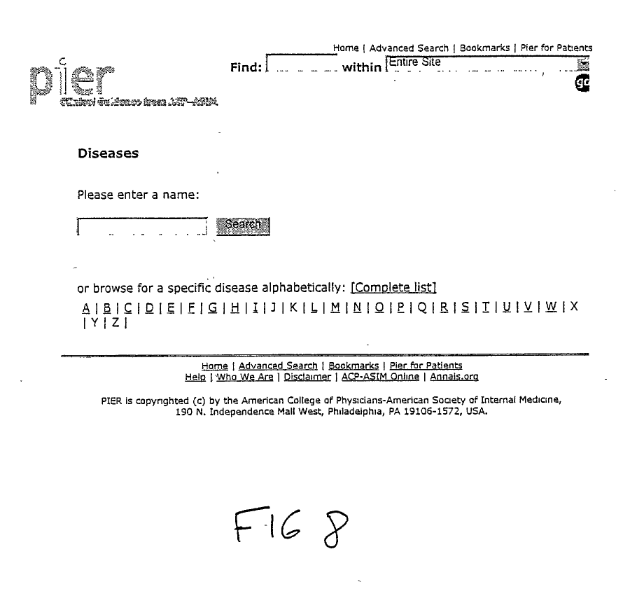

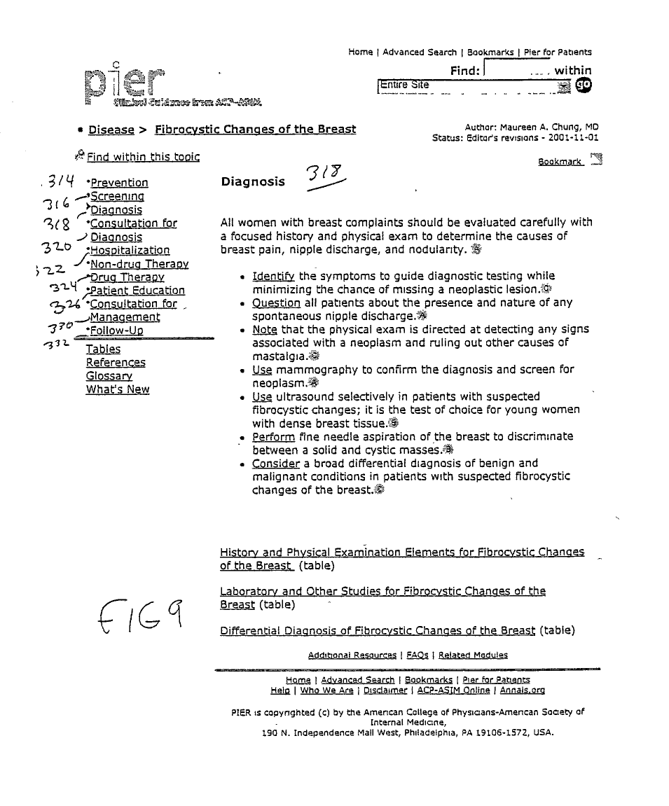

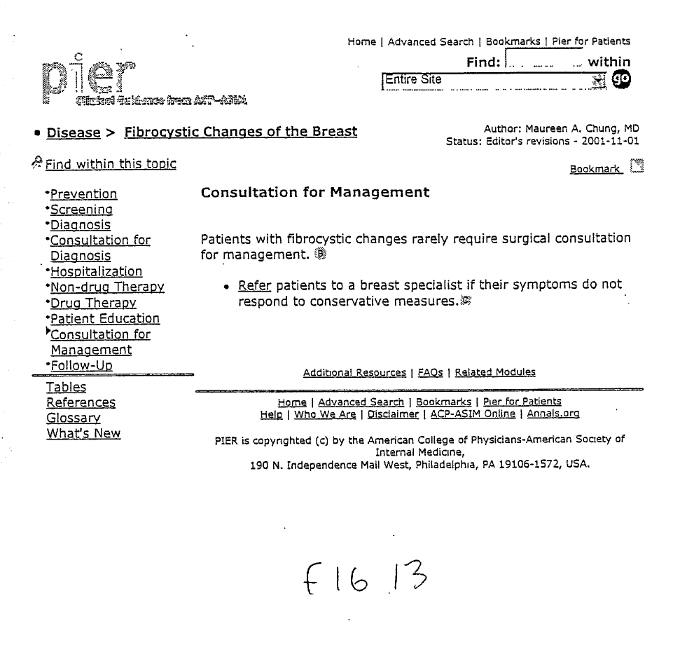

As shown in FIG. 8, a disease may be selected by calling to an initial screen which provides an alphabetized listing of various diseases. The disease “fibrocystic changes of the breast” is selected as exemplary. As shown in FIG. 9, this selection incorporates a series of subcategories including prevention 314, screening 316, diagnosis 318, consultation for diagnosis 320, hospitalization 322, non-drug therapy 324, drug therapy 326, patient education 327, consultation for management 330 and follow up 340. Each of the subcategories has its own page, See FIGS. 13-19.

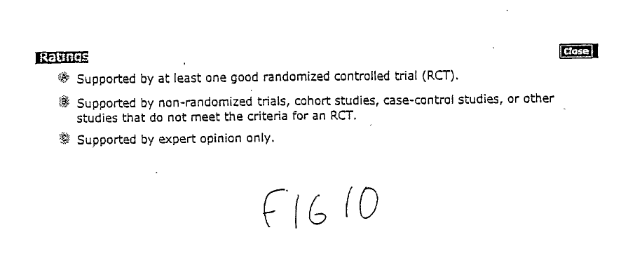

FIG. 9 illustrates the diagnosis for the page for “fibrocystic changes of the breast”. The website further incorporates a rating system for all information provided on the site as shown in FIG. 10. For example, as shown, an A rating means that a reference is supported by at least one good randomized control trial (RCT). A “B” rating means that a reference is supported by non-randomized trials, cohort studies, case-controlled studies, or other studies that do not meet the criteria for an RCT. A “C” Rating means the reference is supported by expert opinion only.

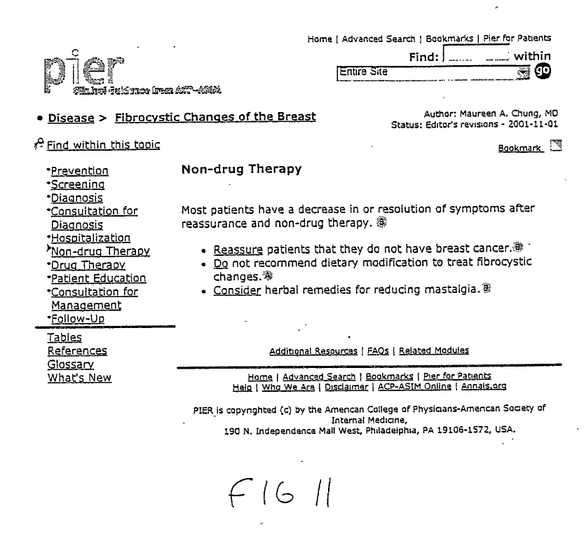

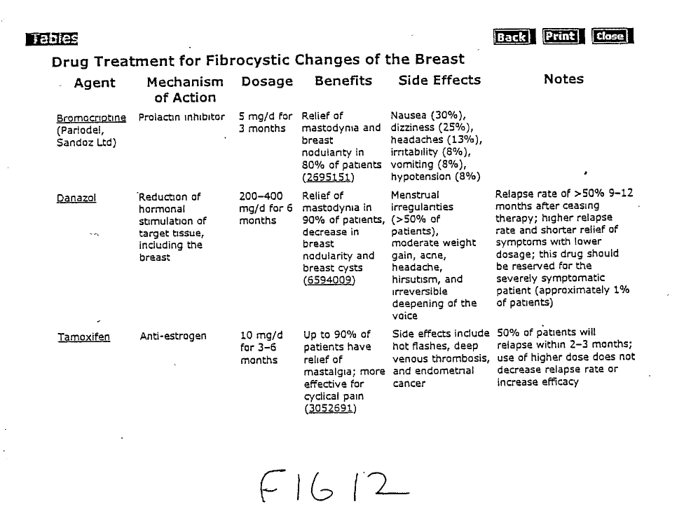

As shown in FIG. 11, non-drug therapy regimens are further shown and disclosed. The website illustrates and shows a large amount of information with respect to specific drug regimens and treatments. As shown in FIG. 12, this includes the agents, the mechanism of action, required dosage, benefits, side effects and notes. Appendices on various other drugs are further shown which thereby provide the medical practitioner with comprehensive information relating to a particular drug. In addition, a set of references is included as well as a glossary.

Accordingly, the present invention provides a comprehensive system whereby medical information and guidance can be provided to a physician. It is to be appreciated that the information and system of the present invention can be accessed via other mediums and modalities. It can be linked to other content and web information providers. Most importantly, this present invention can be used in conjunction with electronic medical records where it can be linked as dedicated content and guidance.

To summarize, unlike traditional medical information sources, the present invention is centered on specific, bottom-line action statements with links to explanations, sources, rationale, etc. With traditional medical information sources, specific, bottom-line action statements, if they exist at all, are buried in pages of discussion and commentary. In the present invention, content will typically be tagged with codes from several controlled terminologies that are used routinely in healthcare systems. This enables linking directly from the institution's systems to patient-appropriate action statements without requiring the user to instigate a search. The basic structure of the system is identical from topic to topic. Information structure is organized to match the clinical workflow. A critical advantage of the present invention is its speed: the physician gets to the relevant bottom-line action statements faster and with significantly less cognitive overhead.

The present invention has been described with reference to the above-discussed preferred embodiments. It is to be recognized that other embodiments fulfill the spirit and scope of the present invention and that the true nature and scope of the present invention is to be determined with reference to the claims attached hereto.

1. A hierarchical system for providing medical information comprising:
a control unit for setting forth navigation and for providing a plurality of medical guidance information for use by a medical professional;
means for assisting the medical professional in navigating and searching disease specific hierarchical information in a database;
means for distributing the hierarchical information to said medical practitioner; and
means for rating the hierarchical information.

2. A hierarchical system for providing medical information to medical practitioner comprising:
a control unit for setting forth system navigation and for providing a plurality of rated medical guidance information stored in a database;
means for assisting a medical professional in obtaining disease specific hierarchical information from the database; and
means for distributing hierarchical information to said medical practitioner.

3. A method for generating medical information in a global computer system comprising the following steps:
authoring a plurality of medical related guidance statements related to specific diseases and rating said information based upon at least one reliability criterion;
placing the plurality of statements in an accessible database as a module and linking thereto a plurality of additional information and data related to the guidance statements; and
providing end users with access to statements and modules via a user interface.

4. The method of claim 3 wherein each module corresponds to a specific disease.

5. The method of claim 3 where said additional information comprises both drug related and non-drug related information.

6. A method for generating medical information for use in a global computer system comprising the following steps:
authoring and pre-storing a plurality of medical related guidance statements related to specific diseases and providing for each a qualitative rating based upon the evidence supporting the statement;
placing the guidance statements within an accessible database as a module and linking thereto a plurality of additional information and data related to the guidance statements; and
providing end users with access to statements and modules via a user interface such that the guidance statements, additional information and data are accessible.
